(19) United States
(12) Patent Application Publication
(10) Pub. No.: US 2026/0096721 A1
(43) Pub. Date: Apr. 9, 2026

(54) Method and device for cleaning medical instruments using modulating compressed gas pulses

(71) Applicant: Hammann GmbH, Landau (DE)

(72) Inventors: Paulina Lämmer, München (DE); Norbert Klein, Grosbliederstroff (FR)

(21) Appl. No.: 19/115,462

(22) PCT Filed: Sep. 26, 2023
(86) PCT No.: PCT/EP2023/076484

§ 371 Date: Mar. 26, 2025

(30) Foreign Application Priority Data
Sep. 30, 2022 (EP) 22199043.5

(51) Int. Cl. A61B 1/12

(52) U.S. Cl. CPC A61B 1/125

(57) ABSTRACT

A method for cleaning medical instruments, which comprise at least one hollow channel to be cleaned, using modulating compressed gas pulses. At least one feed channel is provided upstream of the flushing section of the hollow channel of the medical instrument in order to accelerate the liquid volume in the flushing section. The feed channel is partially filled with a flushing liquid prior to applying the compressed gas pulses, wherein the feed channel is dimensioned with respect to the line geometry, the line diameter, and/or the line length thereof such that liquid blocks can be completely formed within the feed channel upon supplying the compressed gas mixture in order to pass through the line cross-section of the adjoining flushing section so as to completely fill the line.

### TECHNICAL FIELD

The present invention relates to a method and a device for cleaning medical instruments comprising at least one hollow channel to be cleaned by means of modulating pressurized gas impulses.

### STATE OF THE ART

Reusable medical instruments with at least one lumen or hollow channel are used in almost all fields of human, dental and veterinary medicine. Examples of such instruments are endoscopes, gastroscopes, colonoscopes, rectoscopes, proctoscopes, laparoscopes, arthroscopes, thoracoscopes, probes, hoses or catheters.

Examinations with medical instruments are carried out in body orifices that are colonized with microorganisms. As a result, the instruments become contaminated. A contaminated endoscope requires thorough and efficient cleaning (known as “pre-cleaning”) after the examination and before disinfection or sterilization. For reutilization, a hygienic cleaning is thus essential, as an instrument that has not been thoroughly cleaned poses a risk of infection.

The reprocessing of flexible endoscopes and other medical instruments is complex. It usually consists of pre-cleaning as well as subsequent disinfection and conditioning, including drying. While disinfection and conditioning are carried out automatically in devices, the pre-cleaning has so far been carried out manually by medical professionals. This is not particularly economical and difficult to validate. Work steps that are carried out manually entail an increased risk of contamination of patients, staff and the environment.

So far, these instruments have been pre-cleaned manually, in Germany in accordance with the guidelines of the Robert Koch Institute (RKI) and the Federal Institute for Drugs and Medical Devices (BfArM). This manual pre-cleaning is currently carried out using mainly non-foaming cleaners and brushes. The existing pre-cleaning method includes a leak test, external wiping and subsequent immersion in a cleaning bath. Often, the cleaning bath is a multienzyme cleaner bath, although other cleaners are sometimes used instead of enzymes. The cleaning liquid is changed on a daily basis or after heavy soiling. The working channels of an endoscope are cleaned with a brush, wherein the discharge enters the liquid of the cleaning bath. This cleaning liquid is then rinsed through the endoscope channels in the basin. Finally, it is blown with air to essentially dry the surfaces so that dilution of disinfectants is avoided. Only after that, the endoscope is ready for disinfection or sterilization.

Actually, new brushes should be used to pre-clean each channel. However, cleaning brushes used for an endoscope cleaning device are usually subjected to cleaning and disinfection between each application cycle by rinsing them with water, soaking them in an enzyme bath and subjecting them to the same disinfection as the endoscope. The number of application cycles of such cleaning brushes is not specified. Many of them are still used until the bristles broke off and thus the endoscope channels get damaged. Furthermore, the correct brush has to be used for each channel diameter. In addition, not all channels can be brushed as some of them have channel diameters that are too small and there are no brushes available for them.

Manual pre-cleaning poses a considerable risk of contamination for the people carrying out this procedure. It has been shown that the quality of manual pre-cleaning is subject to fluctuations, for example, due to the different users and brushes. As a result, medical devices could be contaminated and therefore pose a risk of contamination for patients. The manual cleaning method requires a high consumption of water and disposables such as gloves and brushes.

Some procedures, such as diagnostic examinations of the human esophagus and stomach, can be completed within 10 to 15 minutes after the patient has been sedated, while pre-cleaning requires approximately 15 minutes and disinfection 40 to 50 minutes. This is unsatisfactory.

DE 10 2004 040 734 B3 and DE 103 21 991 B3 describe a device for cleaning and/or disinfecting hollow bodies, particularly medical hoses, catheters or endoscopes with at least one coupling connected to a water channel for connecting the water channel to the hollow body, wherein a flow constriction in the form of an adjustable nozzle and a pressure sensor are arranged upstream of the coupling in flow direction.

EP 0 71 1 529 A1 describes a method for checking and cleaning instruments for minimally invasive surgery or minimally invasive examination of body cavities. The channel is checked for permeability and, if the flow is too low or lacking, the respective instrument is recognized as not being sufficiently flushable and is registered for rejection.

U.S. Pat. No. 10,722,491 B2 describes a method for cleaning an endoscope in a computer-controlled washer/disinfector in which each lumen of an endoscope is connected to a liquid distribution system to selectively convey pressurized air or pressurized liquids through a lumen in an endoscope.

WO2013037491A1 describes a device for flushing endoscope channels of an endoscope with flushing agent from a flushing agent supply, wherein the device comprises two flushing agent distributors, which are each connected to the flushing agent supply, and a number of flushing channels for introducing flushing agent from the flushing agent supply into a respective endoscope channel. Further, a testing device is provided by means of which an endoscope channel, which is connected to the first flushing agent distributor via one of the flushing channels, can be checked for blockage.

DE69837790T2 describes a method for removing biofilm and debris from the inner surfaces of a pipe/hose comprising guiding a stream of a mixed phase cleaning composition, wherein the composition comprises an aqueous solution of water and a surfactant mixed with a pressurized pulsed gas in order to produce a turbulent mixture or foam. The pressure in the pipe or hose provides a dynamic impact which removes the biofilm and debris. It is further provided that water or pressurized air is flushed through the pipe or hose.

DE 35 02 969 A1 describes a method and a device for cleaning a pipeline by means of simultaneously introduced impulses of a liquid or a gas, wherein these impulses are mixed into overall impulses, which intermittently pass through the pipeline. By doing so, the impulses of the liquid or gas are broken down into individual impulses.

### DESCRIPTION OF THE INVENTION

Against this background, it is therefore the object of the present invention to provide an improved method and a device for fast and efficient (pre-)cleaning of medical instruments that enable fully integrated and automated cleaning and can be validated by reproducible cleaning results in a short time.

This object is solved by an impulse flushing method with the features of claim 1 and a corresponding device for carrying out such a method. Preferred embodiments can be found in the subclaims.

The method according to the invention for cleaning medical instruments, particularly endoscopes, is based on impinging modulating pressurized gas impulses to a hollow channel of the medical instrument, which is partially filled with a flushing liquid to form alternating liquid blocks and gas blocks that are driven in pulses along a flushing section from a feeding point through the hollow channel to a discharging point in order to remove sediments on the walls of the hollow channel. The alternating liquid blocks and gas blocks lead to shear forces on the walls of the hollow channel of the medical instrument, which results in a mechanical removal of sediments or contamination. A hollow channel according to the invention here refers to an entirely closed lumen or a hollow body in a medical instrument, for example an endoscope.

In a first method step, the hollow channel to be cleaned of the medical instrument is coupled in a fluid-tight manner via one or more coupling elements to a pressure impulse device, with which the pressurized air impulses are generated. At the same time, a targeted liquid supply is required to form the liquid blocks. Preferably, an individual coupling element is provided for each hollow channel of the medical instrument. The coupling elements can have different sizes and types so that different medical instruments from different manufacturers can be coupled to the cleaning device or the pressure impulse device.

After coupling the hollow channel of the medical instrument to the coupling elements of the pressure impulse device, the hollow channel is partially filled and subsequently impinged with modulating pressurized gas impulses generated by the pressure impulse device to form alternating liquid blocks and gas blocks that are driven in pulses along a flushing section from a feeding point through the hollow channel to a discharging point in order to remove sediments on the walls of the hollow channel. The flushing liquid can be water, cleaning liquid or a solution, including enzymes, disinfectants or cleaning chemicals.

Although such pressure impulse flushing methods are known, they cannot be used in the field of medical instruments with their hollow channels with small nominal diameters or would not be efficient enough to meet the high hygienic requirements.

To increase the efficiency of cleaning, at least one pre-run channel lor accelerating the volume of the liquid is arranged upstream of the flushing section of the hollow channel of the medical instrument, according to the invention. The pre-run channel serves as an acceleration section for the liquid blocks that form. For this purpose, the pre-run channel is first partially filled with flushing liquid before being impinged with pressurized gas impulses. Preferably, the pressure Impulse device therefore contains connections and ducts for partially filling the pre-run channel. Preferably, partial filling of the pre-run channel with liquid of approximately 10 to 30 % of the pipe volume is sufficient. Preferably, the pre-run channel is partially filled by specifically metering the pressurized gas supply and the flushing liquid supply depending on the impulse duration and interval duration. These control devices are part of the pressure impulse device.

In regard to its pipe geometry, its pipe diameter and/or its pipe length, the pre-run channel is dimensioned in a way that, when impinging the pressurized gas mixture, the liquid blocks can completely develop within the pre-run channel in order to travel through the pipe cross-section of the subsequent flushing section in a pipe-filling manner. By means of the pre-run channel, it is therefore possible to clean medical instruments far more efficiently using pressurized gas impulses. The data obtained by the inventors actually show that when a pre-run channel is arranged upstream of the actual flushing section (cleaning section), the wall shear stress is significantly higher than without such a channel. The pre-run channel for the liquid blocks, which is arranged upstream of the flushing section or cleaning section, has the effect that the liquid blocks can form in a pipe-filling manner before the flushing section of the hollow channel of the medical instrument begins. The pre-run channel thus functions as an acceleration section for the liquid blocks that form and ensures that they have completely formed in a pipe-filling manner before the flushing section begins. The sediments of the hollow channel of the medical instrument present on the channel walls of flushing section are removed due to the developing large shear forces and wall shear stresses.

The pre-run channel arranged upstream of the actual flushing section or the acceleration section thus obtained enables a more efficient disinfection, conditioning and condition assessment. The pre-run channel according to the invention is also characterized in that the energy required for cleaning is significantly reduced compared to conventional impulse flushing methods. The efficiency of the method is also improved by arranging the pre-run channel upstream of the actual flushing section. The method according to the invention is further characterized in that the cleaning of the hollow channels and thus, for example, the pre-cleaning of endoscopes is significantly more hygienic compared to conventional cleaning methods and can be carried out more quickly.

The term “pressurized gas” used herein generally refers to a compressed gas or gas mixture. The term “gas” not only includes pure gases, but also gas mixtures. Preferably, pressurized air is used as pressurized gas in the present invention. However, In general, other (inert) gases or gas mixtures can also be used as pressurized gas, for example argon, nitrogen or carbon dioxide. The pressure level is preferably between 3 and 10 bar,

The term “flushing section” used herein refers to hollow channel section to be cleaned of the hollow channel to be cleaned of the medical instrument and can also comprise the connections.

The term “pre-run channel” as used herein describes a hollow channel section that is arranged upstream of the actual flushing section.

According to the invention, the terms “flushing” and “cleaning” are used synonymously and describe either flushing or cleaning of a hollow channel, wherein one can also require the other.

In preferred embodiments of the impulse flushing method, a leak test is carried out before impinging modulating pressurized gas impulses to the hollow channel. This tests the tightness of the space between the outer sheath of the endoscope and its individual channels. This ensures that cleaning can be carried out correctly and thus reliably hygienically.

In a further preferred embodiment, a pressure test is carried out before impinging modulating pressurized gas impulses to the hollow channel to check the tightness of the space between the endoscope sheath and the outside of the channels lying inside the medical instrument.

In a further preferred embodiment of the pressure impulse method, it is provided that the flushing liquid is sucked in during the pressurized gas impulse and is atomized in the hollow channel to be cleaned via a Venturi nozzle. The pressurized gas impulses distribute the flushing liquid to the inner walls of the device to be cleaned. If the flushing liquid contains additional auxiliary substances (e.g., detergents or enzymes), the exposure time is relatively short due to the concentration of the auxiliary substances in these solutions.

This embodiment can be used for pre-cleaning before the actual impulse flushing cleaning. The structure of any residual contamination remaining after pre-cleaning is changed, so that it can be mobilized and completely removed during the subsequent impulse flushing cleaning. The subsequent impulse flushing cleaning ensures perfectly hygienically clean surfaces. The pressurized gas impulses that are subsequently impinged to the flushing section without water supply dry and condition the inner walls of the hollow channel of the medical instrument.

Alternatively, a storage vessel for the flushing liquid is provided for impinging modulating pressurized gas impulses to the hollow channel, in which a defined initial pressure is set. Preferably, the water and pressurized gas supply is metered via the pre-run channel depending of the impulse and interval duration.

Usually, the impulse flushing method according to the invention is used for a wide variety of medical instruments with at least one hollow channel. Preferably, the medical instrument is an endoscope, a gastroscope, a colonoscope, a rectoscope, a proctoscope, a laparoscope, an arthroscope, a bronchoscope, a thoracoscope, a probe, a tube, a hose or a catheter. The medical instrument can also be permanently installed medical devices. The different areas of application and manufacturers lead to different diameters of the hollow channels to be cleaned. Preferably, the hollow channels to be cleaned of the medical instrument have a diameter of 1 to 20 mm, preferably a diameter of 1 to 7 mm.

Medical devices often have several hollow channels that are contaminated to varying degrees and thus have different cleaning requirements. For hygienically cleaning the hollow channels in such cases, a determined exposure time of the cleaning solution is necessary. Preferably, an exposure interval is provided before or between impinging modulating pressurized gas impulses to the hollow channel, during which a flushing liquid is provided. In preferred embodiments, auxiliary substances, disinfectants, enzymes or chemicals are added to the flushing liquid, as required. The flushing liquid is then incubated in the hollow channel to be cleaned for a determined period of time. In an alternative embodiment, the substances are added to the pre-run channel via a separate supply line. The various hollow channels can be individually controlled. It is thus possible to clean the hollow channel first, add the flushing liquid and allow it to incubate for a sufficient period of time, while the other hollow channels are cleaned during the incubation time. Various solutions can be used for cleaning. Several cleaning sequences can be alternated with incubation times.

The added auxiliary substances are preferably substances for analytically determining residual contamination. For this purpose, different biomarkers are preferably used. In a preferred embodiment, the cleaned hollow channels are then preferably dried with pressurized air.

The auxiliary substances are preferably added via the pre-run channel if further disinfection treatments are planned after pre-cleaning, such as the automatic addition of enzyme solutions or chemical solutions, or the addition of auxiliary substances for analytically determining residual contamination. The supply of such auxiliary substances can preferably be carried out via a separate supply line. After the precise addition of additives such as enzyme solutions or chemical solutions, a recirculation system can be used. For this purpose, a fitting is provided at the discharging point, which conveys the solution to the pre-run channel via a slowly running pump. The solution acts on any residual amounts of contamination remaining after pre-cleaning and changes their structure in a way that they can mobilized and completely removed in the subsequent impulse flushing cleaning.

The impulse flushing method according to the invention enables automated pre-cleaning and is safer and more economical than manual brush cleaning. The cleaning process can be reliably validated and also protects the expensive medical devices.

In addition to the method, the invention also relates to a device for cleaning medical instruments with at least one hollow channel by impinging modulating pressurized gas impulses to the hollow channel to form alternating liquid blocks and gas blocks that are driven in pulses along the flushing section from a feeding point through the pipeline to a discharging point. The device comprises at least one coupling element for coupling the at least one hollow channel to be cleaned to a pressure impulse device, by means of which modulating pressurized gas impulses are generated, which impinges the hollow channel to be cleaned with modulating pressurized gas impulses to form alternating liquid blocks and gas blocks that are driven in pulses along a flushing section from a feeding point through the hollow channel to a discharging point in order to remove sediments on the walls of the hollow channel. The pre-run channel for the liquid blocks, which is arranged upstream of the flushing section, ensures that the liquid blocks can completely form in a pipe-filling manner before the flushing section of the hollow channel of the medical instrument begins.

In order to accelerate the volume of the liquid in the flushing section according to the invention, the device comprises at least one pre-run channel, which is arranged upstream of the flushing section of the hollow channel of the medical instrument. Each pre-run channel can be partially filled with a flushing liquid before being impinged with pressurized gas impulses and in regard to its pipe geometry, its pipe diameter and/or it pipe length, it can be dimensioned in a way that, when impinging the pressurized gas mixture, the liquid blocks can completely form within the pre-run channel in order to travel through the pipe cross-section of the subsequent flushing section in a pipe-filling manner.

The device is preferably equipped with a barcode scanner or an RF-ID scanner for registering a user and/or medical instrument (e.g., endoscope). This makes it possible to register which user is carrying out the cleaning or which medical device is being cleaned. On the one hand, this ensures an accurate documentation and, on the other hand, a program configured for a specific instrument can run automatically. In one embodiment, the device has a touchscreen. The screen guides the user through all process steps and provides feedback. The medical instrument is connected to the device by a coupling element. An illuminated ring around the connection point provides colored feedback. A tank containing cleaning liquid is located underneath.

In a further preferred embodiment, the device comprises sensors. These can be used to measure cleaning parameters, preferably such as pressure, turbidity, electrical conductivity or optical permeability. Furthermore, pressure sensors can be used to check the tightness. This can be carried out after pre-cleaning by measuring the pressure gradient in the closed device at a specified pressure.

The method described above is preferably fully automated and enables a validatable process including pre-cleaning, disinfection and conditioning. Thus, in such an embodiment, the device preferably comprises a control device, which automatically records and adjusts process parameters such as pressure, flow rate, interval duration, interval length.

This type of device for pre-cleaning medical instruments using the impulse flushing method can often be found on sinks. However, it can also be integrated into stationary disinfection devices. Furthermore, it is also possible to equip mobile devices with this technology. This option makes it possible to maintain medical devices on site, for example in case of a disaster, instead of transporting them to existing cleaning facilities, which is time-consuming.

On the one hand, the method and the device according to the invention make it possible to automatically regulate process parameters like cleaning time, water and pressurized gas supply and, if necessary, other auxiliary substances, and on the other hand, to check the condition of the medical instruments to be cleaned already before removing them.

The method and the device according to the invention thus provide an increased safety for users and patients and are considerably more resource-efficient, as cleaning liquids and medical disposables are saved.

The invention is explained in more detail in the following drawings. The invention is by no means limited to the embodiments shown herein. The invention also includes the combination of individual embodiments, individual features or combinations of features.

### DESCRIPTION OF THE INVENTION AND WAYS OF CARRYING OUT THE INVENTION

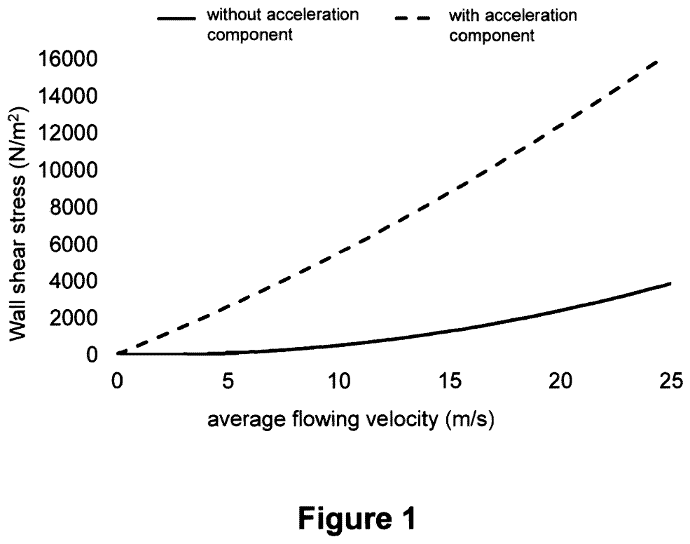

FIG. 1 shows a comparison of the impulse flushing method according to the invention with acceleration component (i.e., with pre-run channel) and without acceleration component (directly connected to the flushing section). The wall shear stress of the flushing section is shown in relation to the average flow velocity. The flow rate of the water blocks is between 15 and 20 m/s during impulse flushing. By arranging the pre-run channel upstream of the flushing section, an acceleration section is created so that the resulting acceleration component significantly increases the wall shear stress.

A decisive advantage of automated cleaning is the significant saving in water consumption of up to 95 %. The lower water requirement and the cleaning time thus considerably reduce the resource requirement and therefore the overall costs.

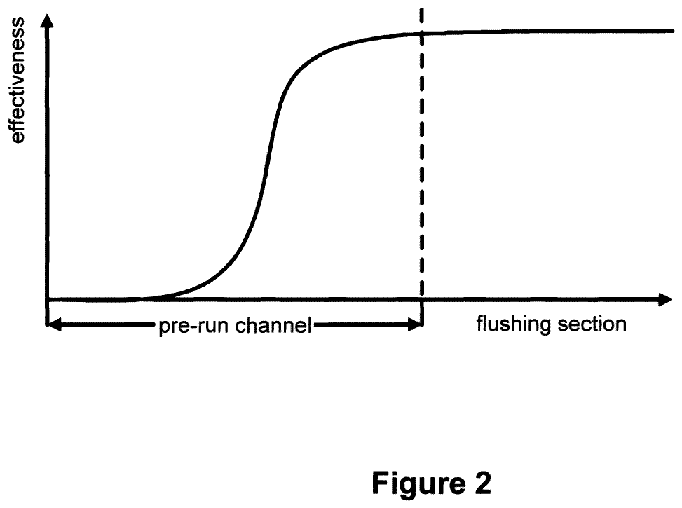

FIG. 2 shows the influence of the pre-run channel on the cleaning success. The effectiveness of cleaning builds up over the course of the pre-run channel and is only fully develop at the beginning of the flushing section. The pre-run channel arranged upstream of the flushing section significantly increases the effectiveness of cleaning compared to conventional impulse flushing without a pre-run channel over the entire cleaning section, as controlled acceleration and formation of the liquid blocks in the pre-run channel is possible.

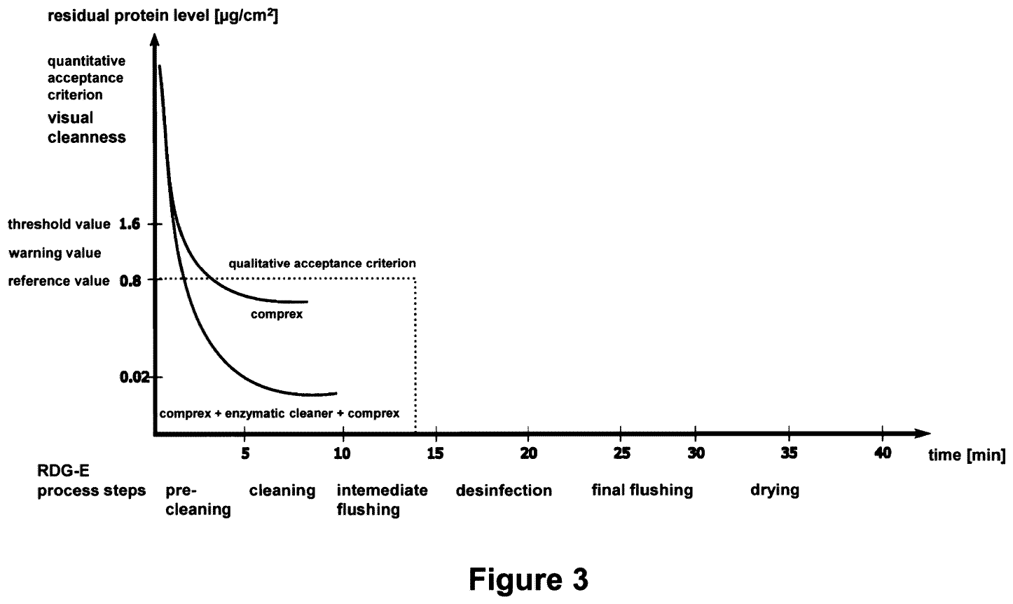

FIG. 3 shows a schematic representation of a comparison of the cleaning performance using the impulses flushing method according to the invention alone and the impulse flushing method according to the invention in combination with an enzymatic cleaner. The residual protein level in μg/cm² in a hollow channel of the medical instrument was measured depending on the time in minutes. The process steps include: pre-flushing, cleaning, intermediate flushing, disinfection, final flushing and drying. The quantitative acceptance criterion is defined in a way that a reference value for a cleaning performance of 0.8 residual protein level in μg/cm² is achieved within 14 minutes (RDG-E, CEN/ISO, 2019. DIN EN ISO 15883-4:2019-06; Washer-disinfectors—Part 4: Requirements and tests for washer-disinfectors employing chemical disinfection for thermolabile endoscopes). By solely using the impulse flushing method according to the invention, the value falls below the reference value of 0.8 μg/cm² in less than 5 minutes. When using the impulse flushing method according to the invention with an enzymatic cleaner, a residual protein level of only 0.02 μg/cm² could be achieved within 5 minutes,

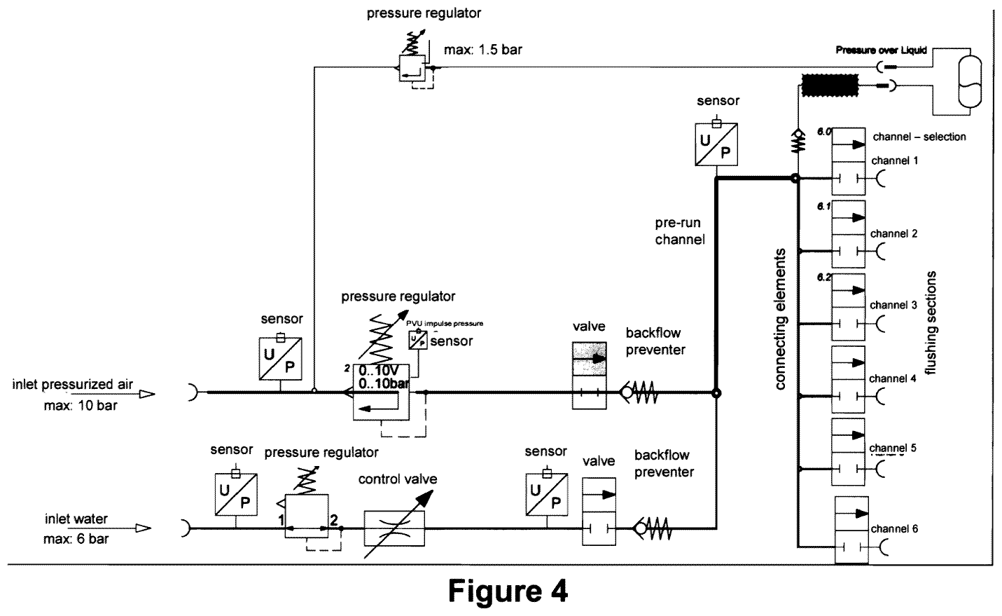

FIG. 4 shows a schematic structure of the device according to the invention and the process sequence. In the embodiment shown, water with a maximum pressure of 6 bar is fed to the pre-run channel at a feeding point. A pressure sensor is integrated to measure the water pressure. A pressure regulator and several valves are provided to regulate the liquid supply. A backflow preventer ensures that the water cannot be retracted through the valve. The control devices and sensors control the partial filling of the pre-run channel, which is essential for carrying out the impulse flushing method.

Pressurized air with a maximum pressure of 10 bar is supplied via a separate inlet. A valve generates modulating pressure impulses to the active pre-run channel. The liquid in the partially filled pre-run channel is accelerated. This creates gas blocks and liquid blocks that are further accelerated within the pre-run channel. The pressure is also regulated via valves. A backflow preventer ensures that the pressurized air cannot return through the valve. The pressurized air impinged to the pre-run channel and the water only mix minimally in the lumen of the pre-run channel. A sensor on the pre-run channel measures the prevailing pressure. The subsequent flushing sections (i.e., hollow channels 1 to 6) of the medical instrument to be cleaned are cleaned by the alternating gas blocks and liquid blocks travelling through the hollow channel.

FIG. 4 also shows that a part of the pressurized air is regulated to a maximum pressure of 1.5 bar in a separate supply line using a pressure regulator. For example, auxiliary substances, disinfectants, enzymes and chemicals are provided in a container. These are preferably pressed into the pre-run channel using pressurized air via a separate supply line. A backflow preventer prevents the mixture from being forced back into the container due to the pressure bubbles generated during the impulse flushing method.

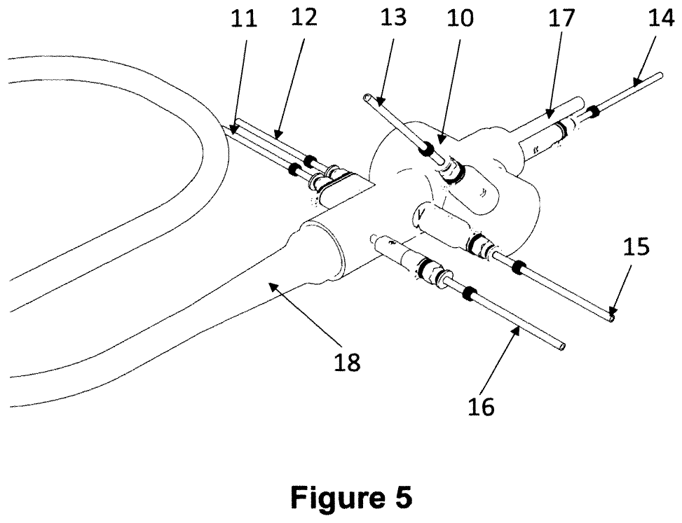

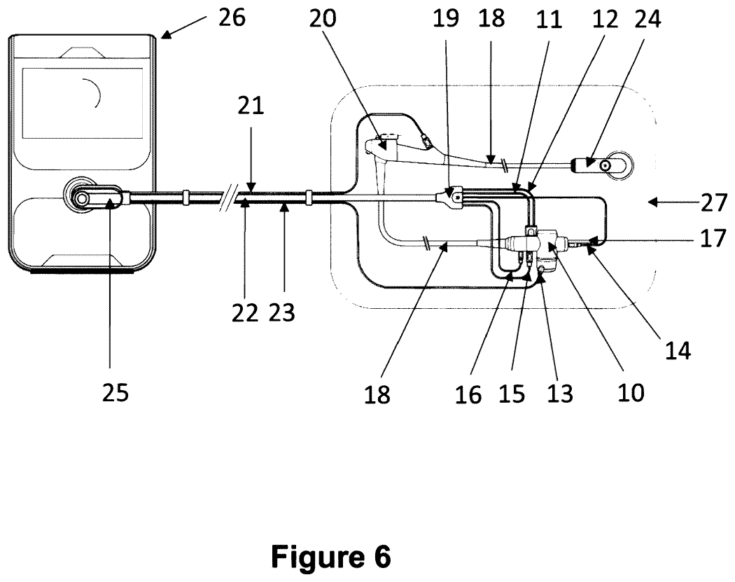

FIG. 5 shows several pipelines 11, 12, 13, 14, 15, 16 with connecting elements for connecting the device to a medical instrument, i.e., in this case an endoscope. The individual pipelines 11, 12, 13, 14, 15, 16 are connected via a supply connector 10. The pipelines 11, 12, 14, 15, 16 with their connecting elements are connected to existing hollow channels to be cleaned and are cleaned. Via these pipelines 11, 12, 14, 15, 16 and their connecting elements, the pressure impulse device 26 is connected to the supply connector 10 of the medical instrument (cf. FIG. 6). By means of the pressurized air impulses generated by the pressure impulse device 26, the connections and hollow channels in the supply hose 18 of the medical instrument are cleaned. As the method and the device are suitable for cleaning several medical instruments from different manufacturers with at least one hollow channel, a variety of different connections is provided that can be connected to the device. An additional existing color coding enables a clear assignment of individual pipelines. The pipeline 13 with its connecting elements is used to connect the pressure impulse device 26 to the pipeline 3 for the leak test. An optical connection 17 for the endoscope is further provided on the supply connector 10.

FIG. 6 shows a schematic structure of the device according to the invention during an endoscope reprocessing. The pressure impulse device 25 is coupled to a medical instrument by means of the coupling element 25 and the pipelines 11, 12, 13, 14, 15, 16 with connecting elements. The medical instrument consists of a control unit 20 for the endoscope, a supply hose 18 and a supply connector 10. The pressure impulse device 26 is connected to air, water and electricity via corresponding connections and supply lines. The medical instrument is connected to the pressure impulse device 26 via a pipeline 21 and a pipeline bundle 22 using a coupling element 25. The individual pipelines 11, 12, 14, 15, 16 are guided in the pipeline bundle 22, which are then divided into five individual pipelines via a distributor 19 and connected individually to the supply connector 10.

The application of modulating pressurized gas impulses and the partial filling with flushing liquid can be optionally carried out via one or more active pipelines. In the embodiment shown, five pipelines 11, 12, 14, 15, 16 are guided in the pipeline bundle 22. The pipeline 21 and the individual pipelines 11, 12, 14, 15, 16 form the individual pre-run channels. Each individual pipeline 11, 12, 14, 15 and 16 connected to the supply connector 10 as well as the pipeline 21 comprise a separate pre-run channel that is independent of the other pipelines. In the embodiment shown, the pre-run channels start at the beginning of the pipelines 11, 12, 14, 15, 16 and 21 on the device side. The pipelines 11, 12, 14, 15, 16 end at the distal end of the pipeline at the supply connector 10. the pipeline 21 ends at the control unit of the endoscope. The liquid blocks are accelerated in the individual pre-run channels by means of pressure impulses generated by the pressure impulse device 26.

The device is suitable for activating a specific pre-run channel for cleaning. For this purpose, the respective pre-run channel, via which cleaning will be carried out, is first partially filled with liquid. The pressure impulses are then impinged by the pressure impulse device 26. The actual flushing section begins at the end of the pre-run channel of the pipelines 11, 12, 14, 15, 16, 21, i.e., cleaning begins in the connected hollow channel of the medical instrument at the latest, and this is where the cleaning efficiency is greatest, as this is where the liquid blocks and pressurized air impulses are strongest. The cleaning efficiency eventually depends on the pre-run channels and the partial filling of the pre-run channel by specifically metering the pressurized gas supply, the flushing liquid supply as well as the impulse duration and interval duration.

By means of the impulse flushing method according to the invention, the individual connecting elements of the supply connector 10 or the control unit 20 of the endoscopes can also be cleaned. The flushing sections start at the connecting elements of the pipelines 11, 12, 14, 15, 16, 21 at the latest and include all hollow channels in the supply hose 18 up to the discharging point 24 at the distal end, which opens into the cleaning basin 27. The pipeline 13 with its connecting element is connected to the pressure impulse device 26 via the coupling element 25 and the channel 23 for the leak test.

The pre-run channels are connected to the medical instrument at different positions, depending on the type and number of hollow channels. As a result, each connecting point of the medical instrument is connected to its own pressure impulse pipeline. The individual flushing sections are controlled via a control device in the pressure impulse device 26.

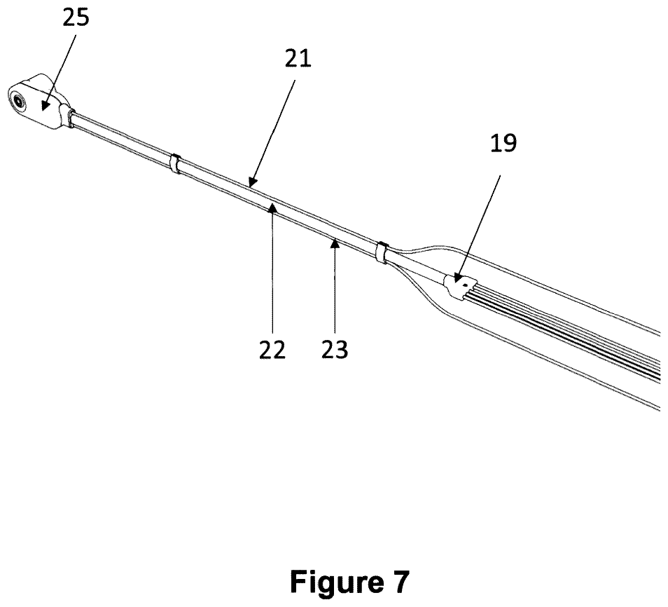

FIG. 7 shows an embodiment of the coupling element 25 according to the invention. On one side, it is connected to a pressure impulse device 26 and connects the device to the hollow channels to be cleaned on the other side. The five pipelines 11, 12, 14, 15, 16 guided in the pipeline bundle 22 and emerging from the distributor 19 can be seen. The pipeline 21 and the pipelines 11, 12, 14, 15, 16 of the pipeline bundle 22, which are divided into individual pipelines by the distributor 19, comprise the pre-run channels for the subsequent flushing sections. Each pre-run channel and flushing section can be separately impinged with pressurized air impulses and can be partially filled with flushing liquid. The partially filled pre-run channel then serves as an acceleration section for the modulating air and liquid blocks that form.

In summary, the method and the device according to the invention enable an automated cleaning of medical instruments with at least one hollow channel, which is carried out faster, more hygienically and with consistent, measurable quality.

### List of References

10 supply connector
11 first pipeline with connecting element
12 second pipeline with connecting element
13 pipeline with connecting element for leak test
14 third pipeline with connecting element
15 fourth pipeline with connecting element
16 fifth pipeline with connecting element
17 optical connection
18 supply hose
19 distributor of the individual channels after the pipeline bundle
20 control unit of the endoscope
21 pipeline with pre-run channel
22 pipeline bundle consisting of the individual pipelines 11, 12, 14, 15, 16
23 channel for leak test
24 discharging point at the distal end
25 coupling element
26 pressure impulse device
27 cleaning basin

1. A method for cleaning medical instruments comprising at least one hollow channel to be cleaned by means of modulating pressurized gas impulses, comprising:
a. coupling of the hollow channel to be cleaned via one or more coupling elements to a pressure impulse device,
b. impinging modulating pressurized gas impulses generated by the pressure impulse device to the hollow channel to form alternating liquid blocks and gas blocks that are driven in pulses along a flushing section from a feeding point through the hollow channel to a discharging point in order to remove sediments on the walls of the hollow channel,
characterized in that at least one pre-run channel for accelerating the volume of the liquid in the flushing section is provided upstream of the flushing section of the hollow channel of the medical instrument, wherein the at least one pre-run channel is partially filled with a flushing liquid before being impinged with the pressurized gas impulses, wherein in regard to its pipe geometry, its pipe diameter and/or its pipe length, the pre-run channel is dimensioned in way that, when impinging the pressurized gas mixture, the liquid blocks can completely form within the pre-run channel in order to travel through the pipe cross-section of the subsequent flushing section in a pipe-filling manner.

2. The method according to claim 1, characterized in that a pressure test is performed before impinging modulating pressurized gas impulses to the hollow channel in order to check the tightness of the space between the endoscope sheath and the outside of the channels lying inside the medical instruments.

3. The method according to claim 1, characterized in that a consistency test is performed before impinging modulating pressurized gas impulses to the hollow channel in order to check the wear of the hollow channel of the medical instrument.

4. The method according to claim 1, characterized in that the flushing liquid is sucked in during the pressurized gas impulses and is atomized in the hollow channel to be cleaned via a Venturi nozzle.

5. The method according to claim 1, characterized in that a storage vessel for the flushing liquid is provided for impinging modulating pressurized gas impulses to the hollow channel, in which a defined initial pressure is set.

6. The method according to one claim 1, characterized in that the hollow channels to be cleaned of the medical instrument have a diameter of 0.5 to 20 mm, preferably a diameter of 1 to 7 mm.

7. The method according to claim 1, characterized in that the pre-run channel is partially filled by specifically metering the pressurized gas supply, the flushing liquid supply as well as the impulse duration and interval duration.

8. The method according to claim 1, characterized in that before or between impinging modulating pressurized gas impulses to the hollow channel, an exposure interval is provided in which a flushing liquid is provided to which auxiliary substances, disinfectants, enzymes or chemicals have been added, as required, and in which the flushing liquid is incubated in the hollow channel to be cleaned for a determined period of time.

9. The method according to claim 1, characterized in that the auxiliary substances, disinfectants, enzymes or chemicals are added to the pre-run channel via a separate supply line.

10. The method according to claim 8, characterized in that the auxiliary substance is a substance for the analytical determination of residual contamination.

11. The method according to claim 1, characterized in that the at least one hollow channel of the medical instrument is dried with pressurized air after cleaning.

12. The method according to claim 1, characterized in that the medical instrument is an endoscope, a gastroscope, a colonoscope, a rectoscope, a proctoscope, a laparoscope, an arthroscope, a bronchoscope, a thoracoscope, a probe, a tube, a hose, a catheter or stationary devices.

13. A device for cleaning medical instruments comprising at least one hollow channel to be cleaned by impinging modulating pressurized gas impulses to the hollow channel. comprising:
a. at least one coupling element (25) for coupling the at least one hollow channel to be cleaned to a pressure impulse device (26)
b. a pressure impulse device (26) used for generating modulating pressurized gas impulses that impinges modulating pressurized gas impulses to the hollow channel to be cleaned to form alternating liquid blocks and gas blocks that are driven in pulses along a flushing section from a feeding point through the hollow channel to a discharging point (24) in order to remove sediments on the walls of the hollow channel,
characterized in that at least one pre-run channel for accelerating the volume of the liquid in the flushing section is provided upstream of the flushing section of the hollow channel of the medical instrument, wherein the at least one pre-run channel can be partially filled with a flushing liquid before being impinged with pressurized gas impulses, wherein in regard to its pipe geometry, pipe diameter and/or pipe length, the at least one pre-run section is dimensioned in a way that, when impinging the pressurized gas mixture, liquid blocks can completely form within the pre-run channel in order to travel through the subsequent flushing section in a pipe-filling manner.

14. The device according to claim 13, characterized in that it comprises a barcode scanner or RF-ID scanner for registering a user and/or endoscope.

15. The device according to claim 13, characterized in that it comprises sensors for measuring cleaning parameters, particularly pressure, turbidity, electrical conductivity or optical permeability.

16. The device according to claim 13, characterized in that a control device is provided to automatically detect and adjust process parameters like pressure, flow rate, interval duration, interval length.
